(19) United States
(12) Patent Application Publication
(10) Pub. No.: US 2016/0235362 A1
(43) Pub. Date: Aug. 18, 2016

(54) INTEGRATED MEDICINE METHOD FOR HEALTH PROMOTION

(71) Applicant: TOTAL WELLNESS CLINIC, TAIPEI CITY (TW)

(72) Inventor: CHEN CHEE LIN, TAIPEI CITY (TW)

(21) Appl. No.: 14/621,728

(22) Filed: Feb. 13, 2015

(51) Int. Cl. A61B 5/00; A61K 36/00

(52) U.S. Cl. CPC A61B 5/4854; A61K 36/00

(57) ABSTRACT

An integrated medicine method for treating and preventing disease, and promoting health is established. The integrated medicine method utilizes western medicine, natural medicine, chinese medicine, functional medicine, and bio-energy/bio-information medicine to assess health and provide treatments and health adjustments. Disease treatment and prevention, and health promotion are provided, combining therapeutic, prophylactic, anti-aging and rejuvenile knowledge to establish an integrated medicine method. With the most advanced medical technology, ideas, products, pathways and networks, advanced types of health care are provided for all people's holistic health through comprehensive physical and mental health assessment using the methods of empirical science, health conditioning, maintenance, and health promotion.

### BACKGROUND OF THE INVENTION

1. Field of the Invention

The present invention relates to medical services. More specifically, the present invention discloses a method for preventing disease and promoting health utilizing integrated medicine techniques, procedures, treatments, and health adjustments.

2. Description of the Prior Art

Conventional medical services are based on disease-based medicine. The level of health of an individual can not be accurately determined prior to detection of a disease in the conventional medical system. As a result, the health of an individual can deteriorate without the individual's knowledge and unknowingly affect their quality of life and lifespan. Importantly, only treating a disease fails to promote health.

Additionally, disease-based treatment typically provides medical services in a departmentalized manner. For example, if a patient complains of lower abdomen pain, the patient is sent to the gastrointestinal department for testing. Not only are the results of the testing typically kept within the department and the patient's record but are also known to a few medical professionals within the department. As a result, other medical departments are not consulted on health conditions that may have influence on the patient's lower abdomen pain. This lowers the effectiveness of treatment and can negatively affect the patient's recovery.

Furthermore, conventional disease-based medical professionals are hesitant to consult other types of medicine such as complementary-alternative medicine (CAM), functional medicine, or natural medicine. As a result, effective alternative therapies, techniques, and treatments are not considered or utilized so the optimal promotion of health can not be achieved.

Therefore, there is need for an efficient system and method for providing total health services to as many people as possible by efficiently expanding medical facilities.

### SUMMARY OF THE INVENTION

To achieve these and other advantages and in order to overcome the disadvantages of the conventional method in accordance with the purpose of the invention as embodied and broadly described herein, the present invention provides an integrated medicine method that utilizes integrated medicine techniques, procedures, treatments, and health adjustments to promote health and prevent disease.

The integrated medicine method of the present invention integrates examination, analysis, diagnosis, health level determination, health adjustment, patient data collection, and treatment techniques from a variety of types of medicine. Incorporating a plurality of medical disciplines allows the present invention to promote predictive, preventive, personalized, and participatory medicine.

Integrated health adjustment procedures are determined and performed with the patient or individual according to the specific individual's health diagnosis. Such health adjustment procedures effectively improve the physical root of the problem and promote the patient's health. The health adjustment of the present invention comprises, for example, nutritional adjustment, weight nutrition, diet adjustment, chiropractic adjustment, exercise, aromatherapy, music therapy, and other methods to provide assistance in the conditioning and promotion of a patient's health.

The integrated medicine method of the present invention does not test for only a positive or negative result but rather builds a complete array of a patient's health. Revisits with additional health evaluation and health adjustment procedures effectively show improvements in the patient's health. Additionally, since the patient or individual actively participates in discussions relating to their level of health and proposed health adjustments and treatments, the patient has more control over the promotion of their health and thereby the treatment is more effective and their health is improved.

Patient data, test data, observation data, analysis data, health reports, health history, health promotion schedule, health adjustment procedures that are performed with the patient and associated data are stored in a patient database that is readily accessible to authorized health professionals at any time.

A health promotion schedule is created and maintained for each patient. The health promotion schedule allows the patient to be notified when a revisit for additional evaluation and health adjustment is due and when a new supply of their health products needs to be obtained. With the aid of the health promotion schedule health professionals can ensure that the patient is following their health improvement program.

Additionally, new medicine and new techniques are reviewed, studied, and tested. If the new medicine or technique is qualified, the new medicine or technique is implemented into the integrated medicine method of the present invention. In this way the integrated medicine method of the present invention is continually expanded and improved.

These and other objectives of the present invention will become obvious to those of ordinary skill in the art after reading the following detailed description of preferred embodiments.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings:

FIG. 1 is a drawing illustrating the structure of integrated medicine for preventing disease and promoting health according to an embodiment of the present invention;

FIG. 2 is a flowchart illustrating an integrated medicine method for preventing disease and promoting health according to an embodiment of the present invention;

FIG. 3A is a flowchart illustrating how to prevent disease and promote health according to an embodiment of the present invention;

FIG. 3B is a flowchart illustrating a study, review, testing, and qualifying new medicine or technique of the integrated medicine method for preventing disease and promoting health according to an embodiment of the present invention;

FIG. 3C is a flowchart illustrating an integrated medicine method for preventing disease and promoting health according to an embodiment of different health status in the present invention;

FIG. 4 is a flowchart illustrating an integrated medicine method for preventing disease and promoting health directed towards anti-aging according to an embodiment of aging factors in the present invention; and

FIG. 5 is a flowchart illustrating an integrated medicine method for preventing disease and promoting health directed towards attention and hyperactivity disorders according to an embodiment of the present invention.

### DESCRIPTION OF THE PREFERRED EMBODIMENTS

Reference will now be made in detail to the preferred embodiments of the present invention, examples of which are illustrated in the accompanying drawings. Wherever possible, the same reference numbers are used in the drawings and the description to refer to the same or like parts.

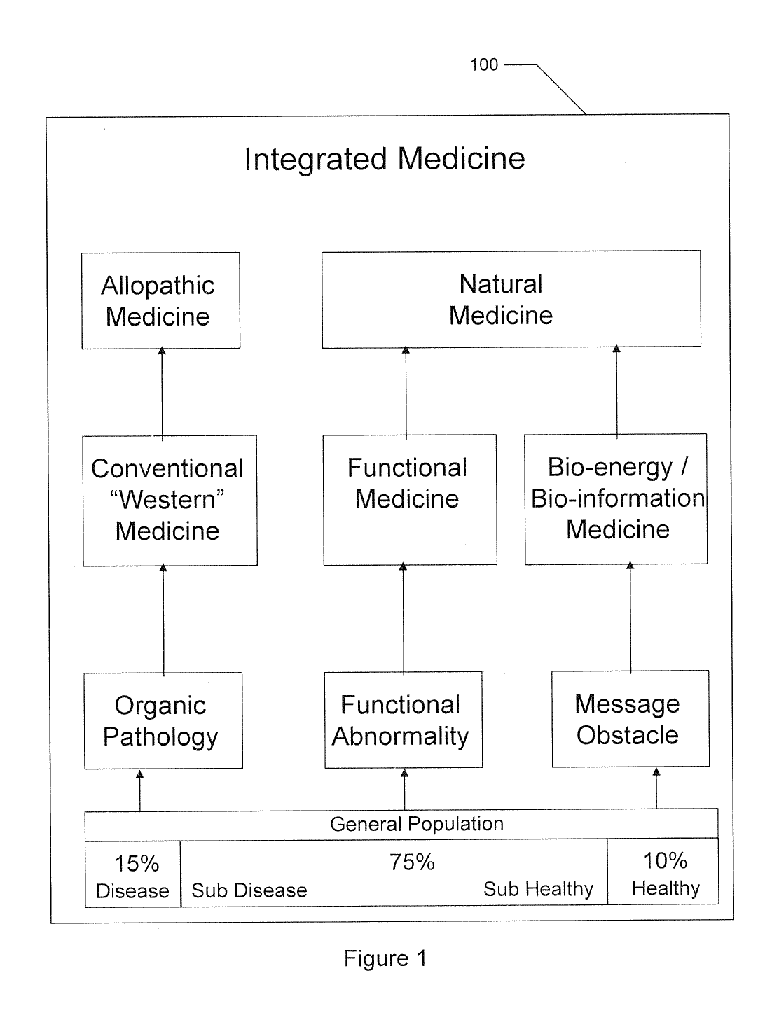

Refer to FIG. 1. The present invention comprises an integrated medicine method 100. In FIG. 1, health status percentages of general population are illustrated on a line from “Disease” (where the patient has been diagnosed with a disease) to “Healthy”. For example, if 100 people are examined, 15% are diagnosed in the Disease category, 10% are diagnosed in the Healthy category, and 75% are diagnosed between the Disease category and the Healthy category. This 75% range from a Sub Disease category to a Sub Healthy category.

Conventional western medicine diagnoses a patient in the Disease category and the disease is determined by organic pathology by testing bodily organs or physiology. Treatment of the disease by conventional western medicine uses a system of medical practice that aims to combat disease by use of remedies, such as drugs or surgery. This system of medicine is also defined as Allopathic medicine.

Bio-energy/Bio-information medicine diagnoses a patient in the Healthy category by observing message obstacles. Bio-energy/Bio-information medicine utilizes its health promotion treatments and those of natural medicine to improve or maintain the patient's health.

Conventional medicine can effectively diagnose and treat patients in the Disease category. However, diagnosis/evaluation and treatment/adjustment of individuals in the Healthy, Sub Healthy, and Sub Disease categories can be difficult since conventional medicine cannot detect the health status of the individuals in these three categories.

To accurately evaluate the health status of individuals in the Sub Disease and Sub Healthy categories, functional medicine observes advanced biochemistry indicators to detect early functional changes and functional abnormality. Functional medicine corrects dysfunction with nutritional supplements and natural medicine to improve the patient's health.

The integrated medicine method 100 of the present invention utilizes western medicine, natural medicine, chinese medicine, functional medicine, and bio-energy/bio-information medicine to assess health and provide treatments.

The present invention provides disease prevention and health promotion, starting with therapeutic, prophylactic, anti-aging and rejuvenile methods to establish an integrated medical health service system. With the most advanced medical technology, ideas, products, pathways and networks, the present invention introduces advanced types of health care to provide comprehensive physical and mental health assessment using the methods of empirical science, health conditioning and health promotion services and management, maintenance, holistic for all people's health.

The integrated medicine method of the present invention uses distinctive equipments, methods such as high-powered blood morphology examination, iridology examination, 3-dimensional bio-energy/bio-information magnetic resonance imaging and functional body analysis (3D MRA), meridian detection, heart rate variability measurement, neurological testing, functional medicine and biochemical testing, spinal structural evaluation, etc.

By providing an integrated medicine health facility utilizing the integrated medicine method, including remote health checks and monitoring, providing the sale of health care products to patients and customers, and receiving referrals from medical professionals that are unable to provide the wide variety of integrated medicine services, the integrated medicine method is not only profitable when a patient is sick (medication, injections, surgery, etc.) but also when an individual is healthy (nutritional supplements, exercise plans, or other health care products). Moreover, the integrated medicine method provides disease treatment and prevention, combats aging, and promotes health and rejuvenation thereby improving the physical and mental health of patients and individuals.

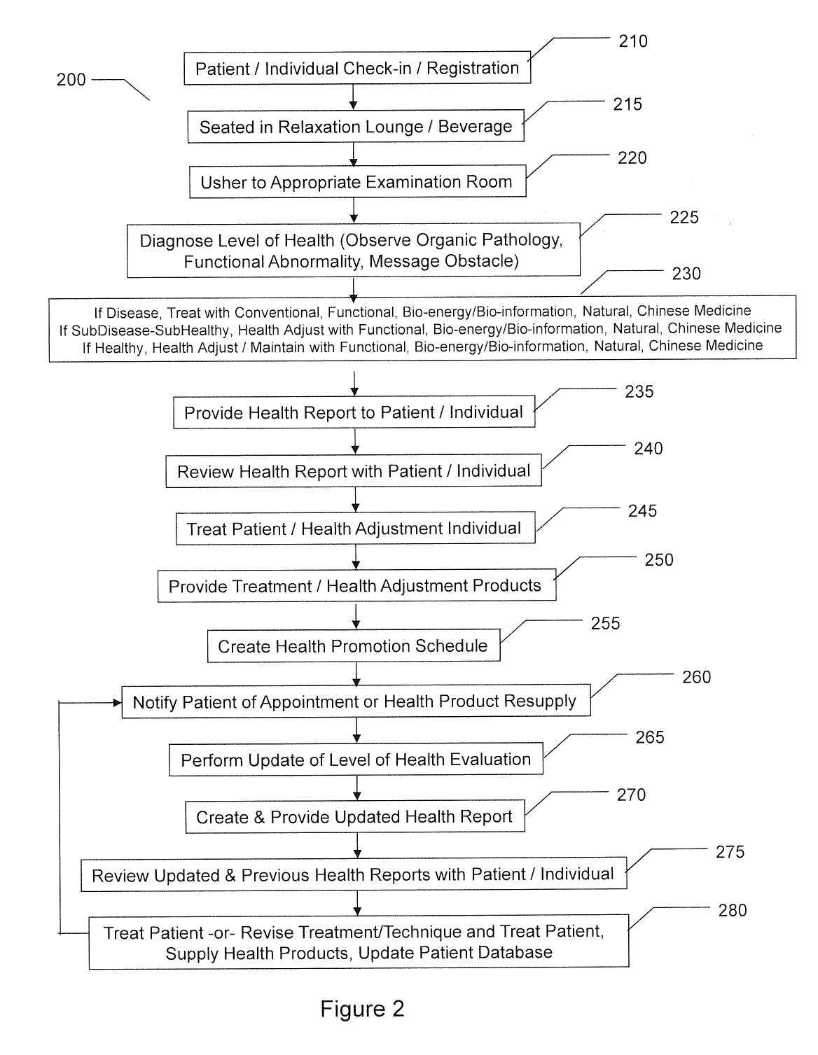

Refer to FIG. 2. The integrated medicine method for preventing disease and promoting health 200 of the present invention comprises a patient or individual checking in at a reception area if they are returning or registering if it is their first visit to the health services facility in Step 210. In Step 215 the patient is shown to a lounge area and provided with a beverage. The lounge area allows the patient to relax and eliminates anxiety and stress prior to their appointment. An advantage of the present invention is that the patient is relaxed for their appointment making diagnosis more accurate since tension, anxiety, and stress symptoms are removed. When time for their appointment the patient is ushered to an appropriate examination room in Step 220.

In Step 225 a health professional performs a variety of examination procedures and diagnoses the patient's or individual's level of health. For example, the health professional utilizes organic pathology by testing bodily organs or physiology, observing functional abnormalities, and observing message obstacles.

In Step 230 the health professional determines the appropriate treatment or health adjustment for the patient or individual. If the patient is diagnosed in the “Disease” category the health professional will plan to treat the patient using techniques and procedures of conventional medicine, functional medicine, bio-energy/bio-bio-information medicine, natural medicine, and chinese medicine. If the individual is diagnosed in the “Sub Disease” to “Sub Health” category the health professional will plan to perform health adjustment procedures using functional medicine, bio-energy/bio-information medicine, chinese medicine, and natural medicine techniques. If the individual is diagnosed in the “Healthy” category the health professional will plan to perform health adjustment procedures using functional medicine, bio-energy/bio-information medicine, chinese medicine, and natural medicine techniques.

In Step 235 a health report is created and provided to the patient or individual. The patient's health report provides patient data and analysis results displayed as text and as visual diagrams. The visual diagrams allow a patient to more easily understand their condition rather than just reading numbers. The clarity of the data encourages the patient to take an active role in improving their health. Proposed treatments and health adjustments are also included in the health report for the patient's consideration. In Step 240 the health professional reviews the health report with the patient, explains the various data contained within the health report, and discusses the proposed treatments or health adjustments.

After discussing the proposed treatments and health adjustment procedures with the patient, the health professional performs the treatments and health adjustment procedures on the patient/individual that the patient or individual has agreed to receive in Step 245. In Step 250 the patient/individual is provided with appropriate health products according to their treatment or health adjustment. The health products comprise, for example, nutritional supplements, exercise plans, exercise equipment, medication, injections, or other health care products.

In Step 255 the health professional creates a health promotion schedule for the patient/individual. The health promotion schedule comprises appointment schedules for when the patient should return and a health product resupply schedule for when the patient should reorder or get a resupply of their health products.

When the health promotion schedule triggers an event, the patient/individual is notified to return for an appointment or resupply their health products in Step 260. Since the health promotion schedule comprises the quantity and dosage of the patient's health products and the patient's treatment schedule, it is known when the supply of health products has been or is close to being used up, or additional diagnosis, treatment, or health adjustments are due. Using the health promotion schedule the health professional can verify whether or not the patient is following the treatment or health adjustment plan.

In Step 265 the patient/individual returns and a diagnosis update is performed to determine the patient's progress. An updated health report is created and provided to the patient/individual in Step 270. The updated health report allows the patient to easily compare his/her patient reports to see the improvement of their health. The patient's health report further comprises graphical depictions showing a patient's history for efficient reference to illustrate the improvement in a patient's health. This allows the patient/individual and health professional to easily see the patient's progress.

In Step 275 the health professional reviews the updated health report along with previous health reports and discusses proposed treatments or health adjustment procedures with the patient/individual.

After review and discussion of the health report with the patient/individual, in Step 280 the health professional treats the patient using an adjusted/revised treatment or health adjustment technique or treats the patient using the previous prescribed treatment or health adjustment technique. Step 280 also comprises updating a patient database. The patient data, test data, observation data, analysis data, health reports, health history, health promotion schedule, health adjustment procedures that are performed with the patient and associated data with the procedures are stored in a patient database. The patient database is stored on a server comprising a microprocessor and a non-transitory memory. Step 280 also comprises supplying or resupplying the patient's health products.

The integrated medicine method returns to Step 260 and continues looping with the notification, diagnosis, updated health reports, updating the patient database, and tuning of a patient's treatment or individual's health adjustment procedures.

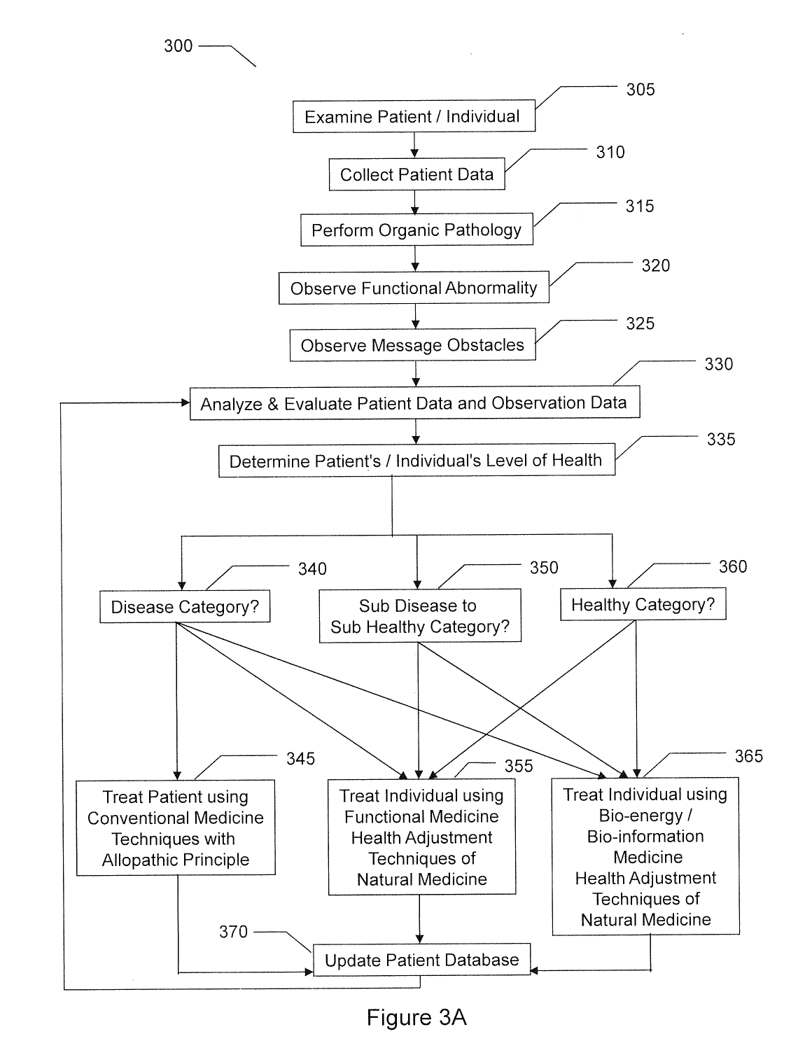

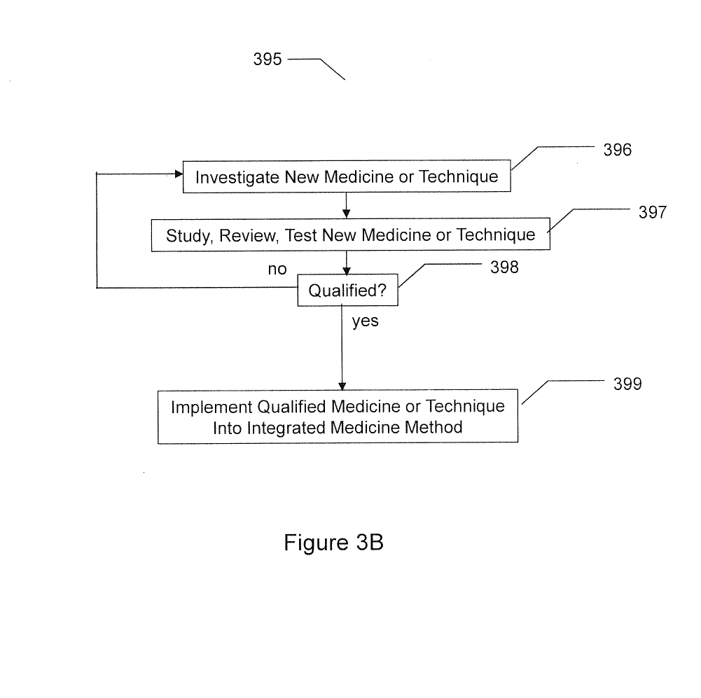

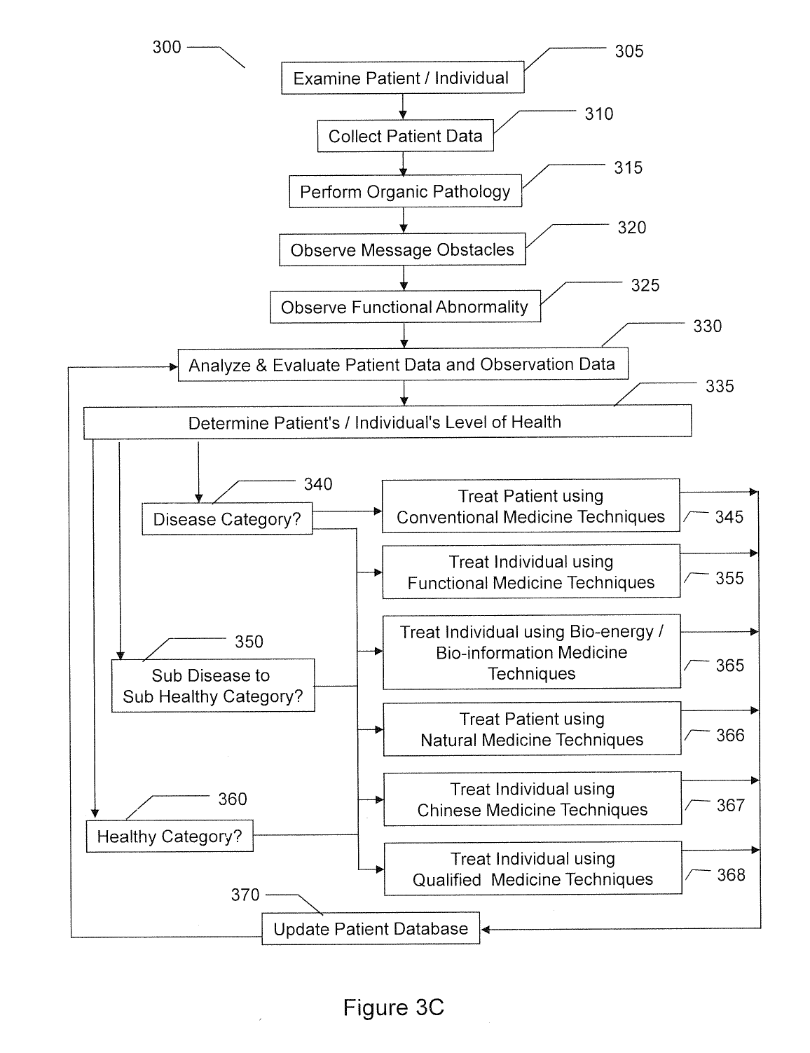

Refer to FIG. 3A which illustrates details of the examination, observation, diagnosis, and treatment processes. The integrated medicine method for preventing disease and promoting health 300 of the present invention comprises examining a patient/individual in Step 305. In Step 310 a health professional collects patient data from a patient/individual. To collect the patient data a plurality of integrated medicine data collection tools is utilized. The data collection tools comprise instruments, implements, devices, or other tools that extract, detect, monitor, or receive data from a patient. This data comprises, for example, heart rate, iris data, blood morphology, blood bio-chemistry data, meridian point data, acupressure data, or 3-dimensional bio-energy/bio-information magnetic resonance imaging and functional body analysis (3D MRA).

In Step 315 the convention medicine technique of organic pathology is performed by testing bodily organs or physiology. In Step 320 the functional medicine technique of observing functional abnormality is performed. In Step 325 the bio-energy/bio-information technique of observing message obstacles is performed.

In Step 330 the collected patient data, test results, observation data are analyzed and evaluated. To assist in the evaluation and analysis process the patient data from the data collection tools, observations, and tests are provided to a plurality of integrated medicine analysis tools. The analyzing tools analyze the data according to various methods or techniques to achieve analysis results. The analyzing tools perform analysis of, for example, blood, nutritional biochemistry, metabolism, gastrointestinal, parasite, hormone, immune system, heavy metal, hypertension, cholesterol, etc.

In Step 335, the patient data, observation data, test results data, analysis data, and evaluation data are used to determine the level of health and health category of the patient/individual.

If the patient's level of health is categorized in the “Disease” category in Step 340, the patient is treated using conventional medicine techniques mainly with Allopathic principle in Step 345. Treatment of disease by conventional western medicine uses a system of medical practice that aims to combat disease by use of remedies, such as drugs or surgery. In addition to treating the patient using conventional medicine techniques, the patient is treated using functional medicine, bio-energy/bio-information medicine, chinese medicine, and natural medicine techniques in Step 345.

If the individual's level of health is categorized in the “Sub Disease” to “Sub Healthy” category in Step 350, the individual is treated using functional medicine health adjustment techniques in Step 355. In addition to treating the individual using functional medicine techniques, the individual is also treated using bio-energy/bio-information medicine, chinese medicine, and natural medicine techniques in Step 355 to improve the individual's health.

If the individual's level of health is categorized in the “Healthy” category in Step 360, the individual is treated using bio-energy/bio-information medicine health adjustment techniques in Step 365. In addition to treating the individual using bio-energy/bio-information medicine techniques, the individual is also treated using functional medicine, chinese medicine, and natural medicine techniques in Step 365 to improve or maintain the individual's health.

In Step 370 the patient database is updated. The patient data, test data, observation data, analysis data, health reports, health history, health promotion schedule, treatments and health adjustment procedures that are performed with the patient and associated data with the procedures are stored in the patient database. The patient database is stored on a server comprising a microprocessor and a non-transitory memory.

The integrated medicine method for preventing disease and promoting health 300 then returns to Step 330.

By accurately determining the level of health of a patient/individual most effective and appropriate health adjustment procedures are selected and performed with the patient/individual. Such health adjustment procedures effectively improve the physical root of the problem and promote the patient's health. The health adjustment procedures of the present invention comprise, for example, nutritional adjustment, weight nutrition, diet adjustment, chiropractic adjustment, exercise, aromatherapy, music therapy, recreation therapy, hypnosis, yoga, detoxification, flower essence, thermo-therapy, hydro-therapy, 3D MRA, chealation, intravascular He—Ne laser therapy, hormone replacement therapy, and other methods to provide assistance in the conditioning and promotion of a patients health.

Refer to FIG. 3B. The integrated medicine method of the present invention further comprises a qualifying process 395. The qualifying process 395 begins by investigating new medicines and new techniques in Step 396. In Step 397 the new medicine or new technique is reviewed, studied and tested. In Step 398 determination is made to qualify or not qualify the new medicine or technique. If the new medicine or new technique is not qualified, the qualifying process 395 returns to Step 396.

If the new medicine or new technique is qualified in Step 398, the qualified new medicine or new technique is implemented into the integrated medicine method of the present invention in Step 399. The qualified medicine or technique is then utilized in the examination, analysis, evaluation, treatment, and health adjustments of the integrated medicine method. As a result, the integrated medicine method of the present invention is continually expanded and improved.

Refer to FIG. 3C. The examination, observation, diagnosis, processes in this embodiment are similar to the embodiment described in regard to FIG. 3A.

If the patient's level of health is categorized in the “Disease” category in Step 340, the patient is treated using conventional medicine techniques in Step 345. Additionally, where appropriate the patient is treated using functional medicine techniques in Step 355, using bio-energy/bio-information medicine techniques in Step 365, using natural medicine techniques in Step 366, using chinese medicine techniques in Step 367, and in Step 368 using qualified medicine techniques that were qualified by the process illustrated in FIG. 3B.

If the patient's level of health is categorized in the “Sub Disease” to “Sub Healthy” category in Step 350, the patient is treated using functional medicine techniques in Step 355, using bio-energy/bio-information medicine techniques in Step 365, using natural medicine techniques in Step 366, using chinese medicine techniques in Step 367, and in Step 368 using qualified medicine techniques that were qualified by the process illustrated in FIG. 3B.

If the patient's level of health is categorized in the “Healthy” category in Step 360, the patient is treated using functional medicine techniques in Step 355, using bio-energy/bio-information medicine techniques in Step 365, using natural medicine techniques in Step 366, using chinese medicine techniques in Step 367, and in Step 368 using qualified medicine techniques that were qualified by the process illustrated in FIG. 3B.

In Step 370 the patient database is updated. The patient data, test data, observation data, analysis data, health reports, health history, health promotion schedule, treatments and health adjustment procedures that are performed with the patient and associated data with the procedures are stored in the patient database. The patient database is stored on a server comprising a microprocessor and a non-transitory memory. The method then returns to Step 330.

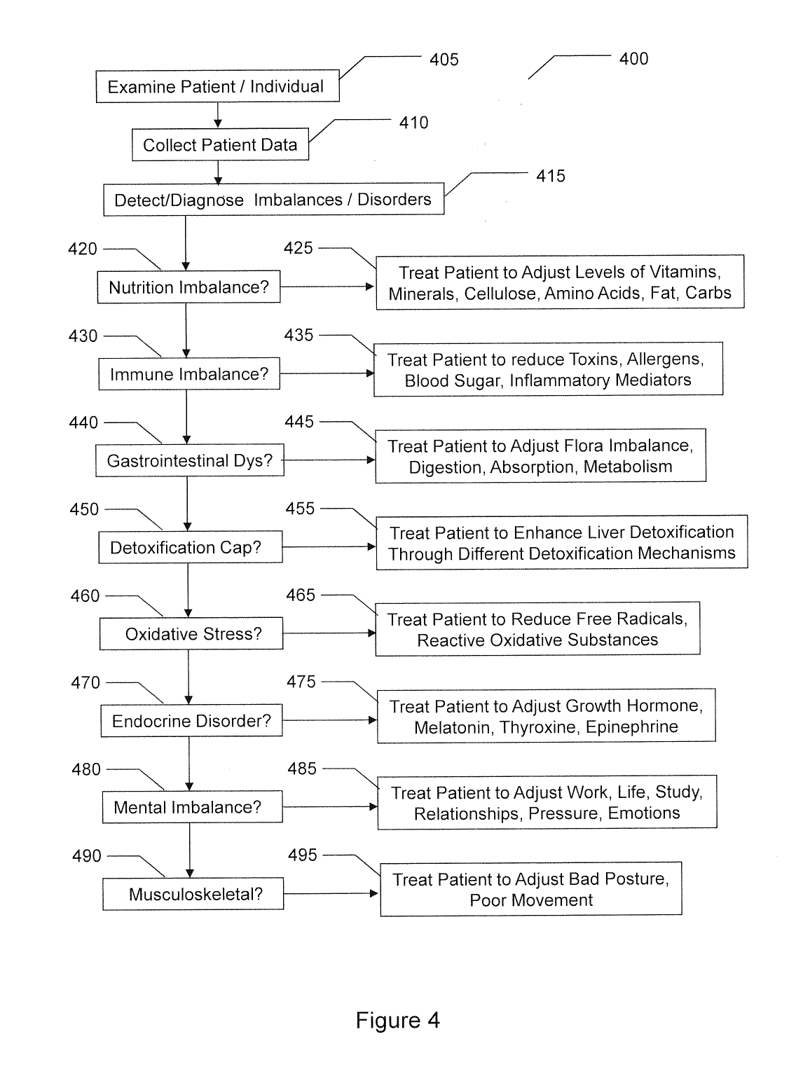

Refer to FIG. 4. The embodiment illustrated in FIG. 4 is directed towards anti-aging. The integrated medicine method for preventing disease and promoting health 400 of the present invention comprises examining a patient/individual in Step 405.

In Step 410 a health professional collects patient data from a patient/individual. For patient data collection, the integrated medicine method of the present invention comprises a plurality of integrated medicine data collection tools for collecting and inspecting a wide variety of data from patients. The plurality of integrated medicine data collection tools comprises, for example, blood collecting tools and devices for extracting blood samples from a patient, Heart Rate Variability (HRV) tools for detecting autonomic nerve function of a patient, meridian detector tools for measuring the bio-energy level of meridian points of a patient, Iris detector tools for inspecting iris texture, color, spots, rings, cracks, or pits. DNA collector tools for obtaining blood samples of a patient's DNA, High Magnification Blood Imaging Tools for optical amplification to collect data of a patient's nutritional status, uric acid metabolism, immunity, and cardiovascular circulation, 3D MRA tools for non-invasive detection of internal conditions using magnetic resonance signals, and other patient data collecting tools.

In Step 415 the collected patient data is analyzed in order to detect and diagnose imbalances, disorders, dysfunctions that are adversely affecting the patient's health and longevity. For analysis, detection, and diagnosis, the patient data collected by the plurality of data collection tools is transferred to a plurality of analysis tools. The plurality of analysis tools analyzes appropriate patient data according to the particular analysis tool. The plurality of analysis tools comprises, for example, a blood analyzer, a biochemical analyzer, a bio-energy analyzer, a nutritional analyzer, a meridian analyzer, and a gastrointestinal analyzer, and other analyzing tools.

The data collection tools and the analysis tools cooperate to identify a patient's health by performing biochemical analysis of, for example, blood, urine, liver and gall bladder function, nutrition, cholesterol, electrolytes, cardiac function, cancer screening, amino acid balance, metabolism, gastrointestinal system, flora balance, protein, pylori bacilli antigen, adrenal stress, sexual hormone, immune function, food allergen, detoxification function, cardiovascular system, brain degeneration risk factor, trace element balance, thyroid hormone, bone loss, hypertension, diabetes, obesity, stress, depression, anxiety, osteoporosis, aging, asthma, etc.

In Step 420 if the patient has been diagnosed with nutritional imbalances, a health professional treats the patient to adjust the level of vitamins, minerals, cellulose, amino acids, fat, carbohydrates, etc. in Step 425.

In Step 430 if the patient has been diagnosed with immune and inflammatory disorders, a health professional treats the patient to reduce the level of a variety of internal and external toxins, allergens, pro-inflammatory substances, oxidative stress, blood sugar regulation disorders, chronic inflammatory mediators, etc. in Step 435.

In Step 440 if the patient has been diagnosed with gastrointestinal dysfunction, a health professional treats the patient to adjust the level of flora imbalance, intestinal leakage (leaky gut), digestion, absorption, metabolism, etc. in Step 445.

In Step 450 if the patient has been diagnosed with low detoxification capability, a health professional treats the patient to enhance liver detoxifications through different detoxification mechanisms, etc. in Step 455.

In Step 460 if the patient has been diagnosed with oxidative stress, a health professional treats the patient to reduce the level of free radicals, reactive oxidative substances, and enhance mitochondrial function, etc. in Step 465.

In Step 470 if the patient has been diagnosed with endocrine disorders, a health professional treats the patient to adjust the level of growth hormone, melatonin, thyroxine, epinephrine, etc. in Step 475.

In Step 480 if the patient has been diagnosed with mental imbalance, a health professional treats the patient to adjust the level and balance of work, study, life, relationships, intense pressure, stress, emotions, etc. in Step 485.

In Step 490 if the patient has been diagnosed with musculoskeletal disorder, a health professional treats the patient to adjust bad posture, poor movement, etc. in Step 495.

The integrated medicine method for preventing disease and promoting health 400 of the present invention is repeated as appropriate to monitor, regulate, and improve a patient's or individual's health, slow their aging, and extend their lifespan with good quality.

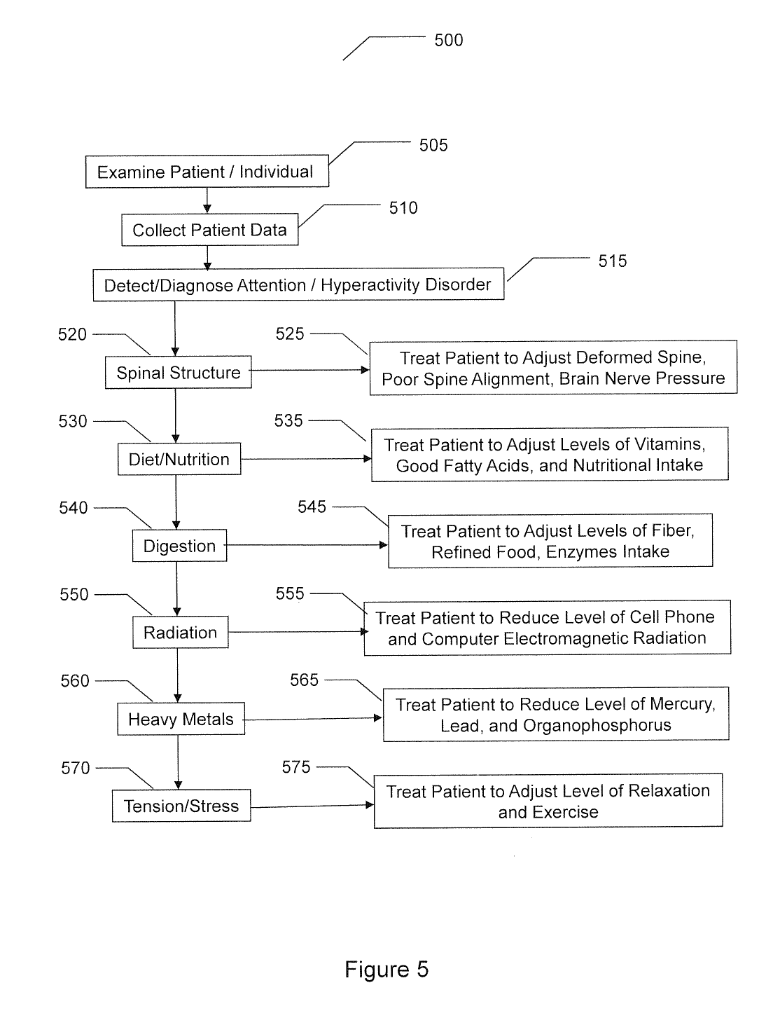

Refer to FIG. 5. The embodiment illustrated in FIG. 5 is directed towards attention disorders and hyperactivity disorders. Instead of medicating the patient as currently done by conventional medicine treatments, the present invention comprises natural medicine, functional medicine, bio-energy/bio-information medicine, and chinese medicine techniques to effectively treat the patient by correcting the root problems of the disorder.

The integrated medicine method for preventing disease and promoting health 500 of the present invention comprises examining a patient/individual in Step 505. In Step 510 a health professional collects patient data from a patient/individual.

In Step 515 the collected patient data is analyzed in order to detect and diagnose attention and hyperactivity imbalances, disorders, dysfunctions that are adversely affecting the patient's health.

If the patient has been diagnosed with a hyperactivity disorder or an attention disorder such as, for example attention deficit hyperactivity disorder (ADHD), the following Steps are performed.

In Step 520 the patient's spinal structure is examined. If the spine is deformed or poorly shaped, the health professional treats the patient by adjusting to reduce the compression of the spine and brain nerve pressure in Step 525.

In Step 530 the patient's diet/nutrition is examined. The health professional treats the patient by adjusting the patient's eating habits, diet, nutritional intake, fruit and vegetable intake, vitamin intake, fish and fish oil intake, good fatty acids intake, etc. in Step 535. Additionally, the health professional provides a dietary schedule and nutritional supplements for the patient.

In Step 540 the patient's digestion and defecation is examined. The health professional treats the patient by reducing refined foods intake and adjusting the intake level of fruits, vegetables, fiber, enzymes, and probiotics to improve gut ecology and function in Step 545.

In Step 550 the patient's exposure to electromagnetic radiation is examined. The health professional treats the patient by reducing the patient's exposure to electromagnetic waves and electromagnetic radiation from mobile phones and computers which affect brain, immune, blood, circulatory and other systems functions in Step 555.

In Step 560 the patient's level of heavy metals and pesticides is examined. The health professional treats the patient by eliminating or reducing the patient's level of heavy metals, pesticides, mercury, and organophosphorus that has accumulated in the body in Step 565. Nutritional supplements are provided to the patient to adjust these levels to reduce hyperactivity.

In Step 570 the patient's level of tension and stress is examined. The health professional treats the patient by adjusting the patient's level of exercise and relaxation using music therapy, regular exercise, flower essence, aromatherapy, etc. in Step 575.

The integrated medicine method 500 is routinely repeated at appropriate intervals to tune the patient's health adjustments and determine the patient's health improvement.

It will be apparent to those skilled in the art that various modifications and variations can be made to the present invention without departing from the scope or spirit of the invention. In view of the foregoing, it is intended that the present invention cover modifications and variations of this invention provided they fall within the scope of the invention and its equivalent.

What is claimed is:

1. An integrated medicine method for preventing disease and promoting health comprising:
examining a patient;
collecting patient data from the patient;
observing organic pathology with the patient data;
observing functional abnormalities with the patient data;
observing message obstacles with the patient data;
analyzing the patient data and observation data;
determining the patient's level of health;
categorizing the patient's level of health;
treating the patient using conventional medicine techniques mainly with allopathic principle, functional medicine techniques, bio-energy/bio-information medicine techniques, natural medicine techniques, and chinese medicine techniques if the patient is categorized in a disease category;
treating the patient using health adjustment techniques of functional medicine, natural medicine, bio-energy/bio-information, and chinese medicine if the patient is categorized in a sub disease to sub healthy category;
treating the patient using health adjustment techniques of bio-energy/bio-information medicine, natural medicine, functional medicine, and chinese medicine if the patient is categorized in a healthy category; and
updating a database of the patient with patient data, observation data, level of health category, and treatment data.

2. The integrated medicine method for preventing disease and promoting health of claim 1, further comprising:
analyzing the patient data and observation data to detect imbalances and disorders;
treating the patient to adjust levels of vitamins, minerals, cellulose, amino acids, fat, and carbohydrates if a nutrition imbalance is detected;
treating the patient to eliminate or reduce toxins, allergens, blood sugar, and inflammatory mediators if an immune imbalance is detected;
treating the patient to adjust flora imbalance, digestion, absorption, and metabolism if a gastrointestinal dysfunction is detected;
treating the patient to enhance liver detoxification through different detoxification mechanisms if detoxification capability is detected as low;
treating the patient to reduce free radicals and reactive oxidizing substances if oxidative stress is detected;
treating the patient to adjust growth hormone, melatonin, thyroxine, and epinephrine if endocrine disorder is detected;
treating the patient to adjust work, life, study, relationships, pressure, and emotions if mental imbalance is detected; and
treating the patient to adjust bad posture, poor movement, and spine if musculoskeletal dysfunction is detected.

3. The integrated medicine method for preventing disease and promoting health of claim 1, further comprising:
analyzing the patient data and observation data to detect attention or hyperactivity disorder;
treating the patient to adjust spine compress and brain nerve pressure if abnormal spinal structure and dysfunction is detected;
treating the patient to adjust levels of vitamins, good fatty acids, and nutritional intake if diet and nutrition imbalance is detected;
treating the patient to reduce levels of fiber, refined food, and enzymes intake if digestion dysfunction is detected;
treating the patient to adjust exposure of electromagnetic radiation if radiation imbalance is detected;
treating the patient to eliminate or reduce level of mercury, lead, and organophosphorus if heavy metal imbalance is detected; and
treating the patient to adjust level of relaxation and exercise if tension and stress disorder is detected.

4. The integrated medicine method for preventing disease and promoting health of claim 1, wherein the database is stored on a server comprising a microprocessor and a non-transitory memory.

5. The integrated medicine method for preventing disease and promoting health of claim 1, further comprising:
providing a health report to the patient, the health report displayed in a combination of numbers, graphics, and text.

6. The integrated medicine method for preventing disease and promoting health of claim 5, further comprising:
reviewing the health report with the patient prior to treating the patient, the health report comprising patient data, observation data, level of health category, and proposed treatments.

7. The integrated medicine method for preventing disease and promoting health of claim 1, further comprising:
creating a health promotion schedule for the patient, the health promotion schedule comprising an appointment date and a health products resupply date.

8. The integrated medicine method for preventing disease and promoting health of claim 7, further comprising:
notifying the patient when the health promotion schedule indicates that the appointment date or the health products resupply date is due.

9. An integrated medicine method for preventing disease and promoting health comprising:
examining the patient;
collecting patient data from the patient;
diagnosing a level of health of the patient;
providing a health report to the patient;
reviewing the health report with the patient;
treating the patient using conventional medicine, functional medicine, bio-energy/bio-information medicine, natural medicine, and chinese medicine if the level of health is disease;
treating the patient using functional medicine, bio-energy/bio-information medicine, natural medicine, and chinese medicine if the level of health is sub disease to sub healthy;
treating the patient using bio-energy/bio-information medicine, functional medicine, natural medicine, and chinese medicine if the level of health is healthy;
providing health products appropriate with treatment to the patient;
creating a health promotion schedule for the patient;
notifying the patient of appointment when revisit is due;
notifying the patient when a new supply of health products is due; and
updating a database of the patient.

10. The integrated medicine method for preventing disease and promoting health of claim 9, further comprising:
analyzing the patient data to detect imbalances and disorders;
treating the patient to adjust levels of vitamins, minerals, cellulose, amino acids, fat, and carbohydrates if a nutrition imbalance is detected;
treating the patient to eliminate or reduce toxins, allergens, blood sugar, and inflammatory mediators if an immune imbalance is detected;
treating the patient to adjust flora imbalance, digestion, absorption, and metabolism if a gastrointestinal dysfunction is detected;
treating the patient to enhance liver detoxification through different detoxification mechanisms if detoxification capability is detected as low;
treating the patient to eliminate or reduce free radicals and reactive oxidizing substances if oxidative stress is detected;
treating the patient to adjust growth hormone, melatonin, thyroxine, and epinephrine if endocrine disorder is detected;
treating the patient to adjust work, life, study, relationships, pressure, and emotions if mental imbalance is detected; and
treating the patient to adjust bad posture, poor movement, and spine if musculoskeletal dysfunction is detected.

11. The integrated medicine method for preventing disease and promoting health of claim 9, further comprising:
analyzing the patient data to detect attention or hyperactivity disorder;
treating the patient to adjust spine compression and brain nerve pressure if spinal structure dysfunction is detected;
treating the patient to adjust levels of vitamins, good fatty acids, and nutritional intake if diet and nutrition imbalance is detected;
treating the patient to adjust levels of fiber, refined food, and enzymes intake if digestion dysfunction is detected;
treating the patient to reduce exposure of electromagnetic radiation if radiation imbalance is detected;
treating the patient to eliminate or reduce level of mercury, lead, and organophosphorus if heavy metal imbalance is detected; and
treating the patient to adjust level of relaxation and exercise if tension and stress disorder is detected.

12. The integrated medicine method for preventing disease and promoting health of claim 9, wherein the database is stored on a server comprising a microprocessor and a non-transitory memory.

13. The integrated medicine method for preventing disease and promoting health of claim 9, wherein the health report is displayed in a combination of numbers, graphics, and text.

14. The integrated medicine method for preventing disease and promoting health of claim 9, wherein the health report comprises patient data, analysis data, level of health category, and proposed treatments.

15. The integrated medicine method for preventing disease and promoting health of claim 9, wherein the health promotion schedule comprises an appointment date and a health products new supply date.

16. The integrated medicine method for preventing disease and promoting health of claim 15, wherein the patient is notified when the health promotion schedule indicates that the appointment date or the health products new supply date are due.

17. The integrated medicine method for preventing disease and promoting health of claim 9, further comprising before examining the patient:
checking in the patient; and
seating the patient in a relaxing lounge and providing the patient with a healthy beverage, the relaxing lounge and healthy beverage allowing the patient to relax and reduce anxiety and stress prior to examination.

18. An integrated medicine method for preventing disease and promoting health comprising:
checking in a patient;
seating the patient in a relaxing lounge and providing the patient with a healthy beverage, the relaxing lounge and the healthy beverage allowing the patient to relax and reduce anxiety and stress prior to examination;
ushering the patient to an appropriate examination room;
examining the patient;
collecting patient data from the patient;
diagnosing a level of health of the patient;
categorizing the level of health of the patient;
proposing treating the patient using conventional medicine, functional medicine, bio-energy/bio-information medicine, natural medicine, and chinese medicine if the level of health is categorized as disease;
proposing treating the patient using functional medicine, bio-energy/bio-information medicine, natural medicine, and chinese medicine if the level of health is categorized as sub disease to sub healthy;
proposing treating the patient using functional medicine, bio-energy/bio-information medicine, natural medicine, and chinese medicine if the level of health is categorized as healthy;
providing a health report to the patient, the health report comprising patient data, level of health category, and proposed treatment displayed in a combination of numbers, graphics, and text;
reviewing the health report with proposed treatment with the patient;
treating the patient with the proposed treatment;
providing health products appropriate with the proposed treatment to the patient;
creating a health promotion schedule for the patient, the health promotion schedule comprising an appointment date and a health products resupply date;
notifying the patient of appointment when the appointment date is due;
notifying the patient when the health products resupply date is due;
re-examining the patient during the appointment;
performing an update of diagnosis of level of health;
providing an updated health report with updated proposed treatment to the patient;
reviewing the updated health report and previous health reports with the patient;
treating the patient with the updated proposed treatment;
providing health products appropriate with the updated proposed treatment to the patient;
updating a database of the patient; and
storing the database of the patient on a server comprising a microprocessor and a non-transitory memory, the database comprising patient data, examination data, level of health data, treatment data, health reports, health products data, and health promotion schedule data.

19. The integrated medicine method for preventing disease and promoting health of claim 18, further comprising:
analyzing the patient data to detect imbalances and disorders;
treating the patient to adjust levels of vitamins, minerals, cellulose, amino acids, fat, and carbohydrates if a nutrition imbalance is detected;
treating the patient to reduce toxins, allergens, blood sugar, and inflammatory mediators if an immune imbalance is detected;
treating the patient to adjust flora imbalance, digestion, absorption, and metabolism if a gastrointestinal dysfunction is detected;
treating the patient to enhance liver detoxification through different detoxification mechanisms if detoxification capability is detected as low;
treating the patient to eliminate or reduce free radicals and reactive oxidizing substances if oxidative stress is detected;
treating the patient to adjust growth hormone, melatonin, thyroxine, and epinephrine if endocrine disorder is detected;
treating the patient to adjust work, life, study, relationships, pressure, and emotions if mental imbalance is detected; and
treating the patient to adjust bad posture, poor movement, and spine if musculoskeletal dysfunction is detected.

20. The integrated medicine method for preventing disease and promoting health of claim 18, further comprising:
analyzing the patient data to detect attention or hyperactivity disorder;
treating the patient to adjust spine compression and brain nerve pressure if spinal structure dysfunction is detected;
treating the patient to adjust levels of vitamins, good fatty acids, and nutritional intake if diet and nutrition imbalance is detected;
treating the patient to adjust levels of fiber, refined food, and enzymes intake if digestion dysfunction is detected;
treating the patient to reduce exposure of electromagnetic radiation if radiation imbalance is detected;
treating the patient to eliminate or reduce level of mercury, lead, and organophosphorus if heavy metal imbalance is detected; and
treating the patient to adjust level of relaxation and exercise if tension and stress disorder is detected.
